(19) United States
(12) Patent Application Publication
(10) Pub. No.: US 2025/0256089 A1
(43) Pub. Date: Aug. 14, 2025

(54) IMPLANTABLE MEDICAL DEVICE AND OPERATION METHOD OF SUCH DEVICE

(71) Applicant: BIOTRONIK SE & Co. KG, Berlin (DE)

(72) Inventors: Brian M. TAFF, Portland, OR (US); Kurt SWENSON, Corvallis, OR (US); David MILLER, Lake Oswego, OR (US); Brad MCMILLAN, Lake Oswego, OR (US)

(73) Assignee: BIOTRONIK SE & Co. KG, Berlin (DE)

(21) Appl. No.: 18/856,963

(22) PCT Filed: Apr. 4, 2023
(86) PCT No.: PCT/EP2023/058804

§ 371 Date: Oct. 15, 2024

Related U.S. Application Data
(60) Provisional application No. 63/335,331, filed on Apr. 27, 2022.

(30) Foreign Application Priority Data
May 20, 2022 (EP) 22174489.9

(51) Int. Cl. A61N 1/02; A61N 1/37; A61N 1/372; A61N 1/375; G16H 40/63

(52) U.S. Cl. CPC A61N 1/025; A61N 1/3706; A61N 1/37254; G16H 40/63; A61N 1/3756

(57) ABSTRACT

An implantable medical device, for example an implantable leadless pacemaker, is disclosed comprising a processor, a therapy signal generator, for example a pacing signal generator, as well as a communication unit and a memory unit, wherein the memory unit is configured to exchange data with the processor, wherein the memory unit comprises a ROM sub-unit, a second memory sub-unit and a state element, wherein the state element is configured to be set to one state of a first state and at least one second state, wherein the second memory sub-unit comprises a RAM, wherein the communication unit is configured to receive information comprising a state input information from an external computing device and to transmit at least one data corresponding to this information to the processor, wherein the processor is configured to set the state of the state element.

### CROSS-REFERENCE TO RELATED APPLICATIONS

This application is the United States National Phase under 35 U.S.C. § 371 of PCT International Patent Application No. PCT/EP2023/058804, filed on Apr. 4, 2023, which claims the benefit of European Patent Application No. 22174489.9, filed on May 20, 2022 and U.S. Provisional Patent Application No. 63/335,331, filed on Apr. 27, 2022, the disclosures of which are hereby incorporated by reference herein in their entireties.

### TECHNICAL FIELD

The present invention is generally directed to an implantable medical device, e.g., an implantable leadless pacemaker, to an operation method of such implantable medical device, a respective computer program product, a computer readable data carrier as well as to a system comprising at least one of such implantable medical device and an external computing device.

### BACKGROUND

Active implantable medical devices (IMDs, implants), for example, a pacemaker (with leads), an implantable cardiac monitor (ICM), an Implantable Leadless Pacer (ILP), an Implantable Leadless Pressure Sensor (ILPS), an Implantable Cardiac Defibrillator (ICD), a subcutaneous ICD (S-ICD) or a neurostimulation device such as a Spinal Cord Stimulation device (SCS device) are configured to collect diagnostics and/or administer therapy signals to the patient, the latter supporting, for example, electrical stimulation of the heart. In service of the noted diagnostic support, such devices notionally contain at least one sensor for collecting physiological signals to monitor the health status of the patient and critical communication unit infrastructure enabling the transmission of such collected signalling (as raw or processed data depending upon the context and application) to an external computing device (e.g., smartphone, programmer, computer, remote server).

A cardiac pacemaker (or artificial pacemaker) is a medical device that generates electrical pulses delivered by electrodes either connected to or fixedly attached to the pacemaker capable of causing the heart muscle chambers (i.e., the atria and/or the ventricles) to contract and, in turn, pump blood. By means of such output these devices replace and/or regulate the function of the patient's cardiac electrical conduction system. One purpose of a pacemaker is to maintain an adequate heart rate (cardiac rate), either as a pro[ for deficiencies in the heart's natural pacemaker, or because a block exists in the heart's electrical conduction system. Additionally, or alternatively, the pacemaker may stimulate different positions within the ventricles to improve their synchronization or provide antitachycardia pacing output to combat life-threatening arrhythmias. Modern pacemakers are programmable by the external computing device (programmer) and allow a health care practitioner (HCP) to select the optimal pacing mode(s) and configuration settings tuned to individual patient needs.

An implantable intracardiac pacemaker (also known as implantable leadless pacemaker-ILP) is a miniaturized pacemaker that is implanted entirely within the blood volume of the patient's ventricle or atrium. ILPs are crucial to the future of cardiac pacing. Aside from core sensing and pacing support, alternative or additional functions of conventional or intracardiac pacemakers include the delivery of electrical or electromagnetic signaling aside from pace output to the heart or its surrounding tissue (e.g., to collect insights on thoracic impedance, for instance) and the assessment of physical body motion or other physiological signals of interest. Due to the highly restricted device size, an ILP has a small battery capacity.

ILP and other small implantable medical devices are challenged in their ability to simultaneously support complex behaviors in a small form factor while still provisioning device longevities aligned with legacy designs. Furthermore, the compact size of such devices, especially subject to long-term residence within the patient create challenges for explantation at end-of-service (EOS). In readily envisionable future embodiments of this miniaturized medical device technology, it is also the case that there is an emerging expectation for therapy to be coordinated across a multitude of implanted medical devices in supporting critical patient needs. Subject to this context, the program setting within these implants, as established by the HCP at the most recent follow-up, demand special handling and caretaking subject to worst-case implant reset dynamics. Worst-case reset behavior amounts to a condition where an active implantable medical device has lost its capacity to function as nominally intended and, as such, administers an “option of last resort” behavior. In a bulk of active implantable medical device designs worst-case resets may occur when the RAM application running on the device has become corrupted in an irreparable manner.

Known medical devices for cardiac rhythm management (CRM) have assumed that a safe response to worst-case reset conditions would always embody a behavior resulting in the delivery of some form of remedial pacing output. This remedial output might not administer the specific mode of therapy best suited for the patient but would amount to a therapy that would keep them supported (even if sub-optimally) till they could connect with their heart care specialist. However, always rendering an output is not known to be a safe condition in contexts where more than a single medical device capable of rendering therapy happens to be implanted with a patient. It is further often the case that worst-case resets of this type are ones that in traditional implanted medical devices such as a pacemaker/ILP have historically applied high amplitude long pulse width pacing output. While such output offers a means to increase the likelihood of effectively engaging with the heart's conduction system, the energy demand associated with such output is not readily aligned with the limited power resources found in ILPs or other small implantable medical devices.

Accordingly, there is the need for an implantable medical device and an operation method for such medical device that increases patient safety in a worst-case reset situation, in particular, if the medical device is a member of a multi-device system or challenges associated with the physical explantation of a device demand that devices approaching their end of service remain stationed in the patient even after a replacement device has been implanted.

The present disclosure is directed toward overcoming one or more of the above-mentioned problems, though not necessarily limited to embodiments that do.

### SUMMARY

At least the above problem is solved by an implantable medical device comprising the features of claim 1, a system comprising the features of claim 6, an operation method of an implantable medical device with the features of claim 8, a computer program product with the features of claim 13 and computer readable data carrier having the features of claim 14.

In particular, at least the above problem is solved by an implantable medical device, for example, an implantable leadless pacemaker, comprising a processor, a therapy signal generator, for example, a pacing signal generator, as well as a communication unit and a memory unit, wherein the memory unit is configured to exchange data with the processor, wherein the memory unit comprises a ROM sub-unit, a second memory sub-unit and a state element, wherein the state element is configured to be set to one state of a first state and at least one second state, wherein the second memory sub-unit comprises a RAM, wherein the communication unit is configured to receive information comprising a state input information from an external computing device and to transmit at least one data corresponding to this information to the processor, wherein the processor is configured to set the state of the state element based on the received data corresponding to state input information, wherein the processor is further configured such that if it identified that at least one pre-defined RAM application has become corrupted in an irreparable manner, the operation of the processor controlling determination of therapy output and transmission of therapy output to the therapy signal generator is based on the data stored in the ROM sub-unit and the present state of the state element.

The implantable medical device with the above features may be an ILP, a conventional pacemaker, an implantable cardiac monitor (ICM), an ILPS, an ICD, a S-ICD, a neurostimulation device or similar.

The implantable medical device comprises a processor, a therapy signal generator, for example, a pacing signal generator, as well as a communication unit and a memory unit. The therapy signal generator, the communication unit and the memory unit are electrically interconnected to the processor and configured to exchange data with the processor.

The processor is generally regarded as a functional unit of the medical device, that interprets and executes instructions comprising an instruction control unit and an arithmetic and logic unit. The processor may comprise or be a microprocessor, a controller, a digital signal processor (DSP), an application specific integrated circuit (ASIC), a field-programmable gate array (FPGA), discrete logic circuitry or any combination thereof. Alternatively, or additionally, the processor may be realized using integrated dedicated hardware logic circuits, in particular, in the case of an ILP due to the small size and extreme power limitation.

The processor processes data and information received from the communication unit and the memory unit. It may further process signal data received from a detector for retrieving physiological signals from the patient, for example, electrical signals of the patient's heart which are detected over time. The signal data determined by the detector and the information received from the external computing device by the communication unit are transmitted to the processor.

The communication unit may be configured to receive information from the external computing device (one-directional communication) or to bi-directionally communicate with the external computing device. The communication may be wireless comprising communication through the body by means of acoustic, conducted, magnetically/inductively coupled or otherwise (generally administered by having a Programmer wand make direct physical contact with the patient) or, in certain contexts (notionally ones other than those used for power-constrained deep implants) over the air via electromagnetic waves, using, for example, MedRadio/MICS/MEDS, EDGE, EV-DO, Flash-OFDM, GPRS, HSPA, LoRaWAN, RTT, UMTS, Narrowband IoT, Bluetooth, WLAN (WiFi), ZigBee, NFC, LTE, Wireless USB, Wibree (BLE), Ethernet or WiMAX in the radio frequency region, or IrDA or free-space optical communication (FSO) in the infrared or optical frequency region. The external computing device (e.g., programmer) is configured to transmit parameters to the medical device by means of a patient/HCP-facing user interface. The input/selection of suitable parameters and transmission of these parameters to the medical device is referred to as programming.

The processor controls determination of a therapy output based on application(s) and parameters provided by the memory unit, and, if applicable, additionally based on signal data received from the detector, and transmits the therapy output to the therapy signal generator which produces the therapy signal based on the therapy output and provides this signal to the patient (e.g., the patient's tissue). The therapy signal generator generates, for example, electrical or electromagnetically therapy signals based on the therapy output determined by the processor to provide relevant support to the patient.

In particular, in case of a pacemaker or an ILP, the detector may be configured to detect the time-dependent electrical depolarization and repolarization field signals such as an electrocardiogram (ECG) or intracardiac (IEGM). These signals may comprise signals caused by the depolarization of the atria (in the following, the intrinsic atrial signal) and electrical signals caused by the depolarization of the ventricles (in the following, the intrinsic ventricular signal). In the case of an ILP, the intrinsic atrial signal may be a far field electrical signal. The detector may preprocess these data, for example, digitize the signals, filter them and/or amplify them prior to relaying the data to the processor. The processor may receive detector signals, for example, intrinsic ventricular signals and intrinsic atrial signals derived from the electrical signals, for example, the intrinsic atrial signal from the P wave and the intrinsic ventricular signal from the QRS complex.

The medical device being an ILP or a conventional pacemaker may normally be operated in the VDD pacing mode (i.e., a pacing mode in which therapy output of the processor is provided such that the ventricle is stimulated in accordance with detected atrial activity) or a VVI-based pacing mode. In the VDD mode, the pacemaker synchronizes ventricular pacing with the intrinsic atrial signal by means of an AV delay. In a VDD ILP that is implanted in the right ventricle, the atrial contraction information may be detected as a far field electrical signal as indicated above. In the VDD mode, the processor may determine from the intrinsic atrial signal and the intrinsic ventricular signal or the ventricular pacing signal the measured intrinsic cardiac interval which, in turn, corresponds to the intrinsic cardiac rate. The current, measured intrinsic cardiac interval may be used to provide the ventricular pacing control signal containing the ventricular pacing time, which may then be transmitted to the pacing signal generator. Based on the pacing control signal, the pacing signal generator produces the electrical pacing signal(s) to transfer it to the electrodes which apply the signal(s) to the heart's tissue adjacent to the electrode. The pacing signals are pulses that begin at a desired time point and have a desired intensity and duration. Further, the pulse waveform may be varied, e.g., pulse width and its amplitude. Information on the pacing signals, e.g., the ventricular pacing signals, that are necessary to produce the correct pacing signals are provided by the pacing control signal, e.g., by the ventricular pacing control signal, of the processor or by the pacing signal generator itself. In particular, the ventricular pacing control signal provides time information of the pacing signal, i.e., information on when the pacing signal shall immediately be provided to the patient's heart, e.g., the information that pacing shall be provided without further deferment. In one embodiment, the pacing signal is overridden by the patient's intrinsic heart activity (i.e., inhibited) and thus the pacemaker avoids transferring any pace output to the electrodes to facilitate an avoidance of stimulation when it is not needed. VVI modes of operation are likewise supported through the elimination of efforts to detect atrial signaling.

The medical device comprises a memory unit which may include any volatile, non-volatile, magnetic, or electrical media, such as a random access memory (RAM), non-volatile RAM (NVRAM), electrically-erasable programmable ROM (EEPROM), flash memory, or any other memory device, wherein the memory unit comprises, in particular, a read-only memory (ROM) sub-unit, a second memory sub-unit comprising the RAM and a state element. The memory unit saves the parameters and further data that are necessary for the medical device to provide the prescribed therapy to the patient. The parameters and further data are required by the processor during execution of the above and below explained steps. The second memory sub-unit further comprises a RAM program application that is normally used by the medical device and, in particular, by its processor, to control determination of the therapy output and to control transmission of the therapy output to the therapy signal generator and thereby provides the prescribed therapy to the patient.

The state element is a section of the memory unit containing a status information. It may be realized as a portion of the volatile memory space which would include retention in RAM, hardware registers, or otherwise. Preferred embodiments would station the state element in hardware registers outside of ROM and RAM code space as an added safety strategy and such registers would be constructed for robustness against single event upsets (SEU—e.g., cosmic ray bombardment, etc.). This status information is used to control the behavior of the medical device, subject to conditions where the processor has identified that at least one pre-defined RAM application has become corrupted in an irreparable manner. The state element is configured to be set to one state of a first state and at least one second state, for example, exactly one second state. The communication unit is configured to receive information comprising a state input information from the external computing device, e.g., a smartphone, programmer, computer or remote server, and to transmit at least one data corresponding to this information to the processor, wherein the processor is configured to set the state of the state element based on the received data corresponding to state input information. Accordingly, the processor sets the state element to the first state or to the at least one second state. One second state may be the initial state of the state element. The present state of the state element is the state which is most recently set by the processor or the initial state if there was no state setting of the state element from operation start of the medical device. In preferred embodiments, setting of the state element would be supported only during active communication sessions with an external device meaning that the implant itself would not have faculties for configuring or changing the element outside of follow-up. For example, the communication unit and the state element of the medical device employ an interface and are used within the environment of the external computing device through which the HCP may instruct specific, targeted medical devices (of a multitude resident within the patient) to be designated as ones essential for administering therapy or as ones that should be disabled and henceforth deny therapy signal output. In the context of change-out conditions this could simply amount to indicating which medical device was the “old” one and which was the “new” replacement medical device. In a nominal case, the “old” medical device may be transitioned toward a deactivated state where it denied a capacity to render therapy at later points in time and the “new” medical device may be designated as one intended to provide brady symptom management henceforth. Alternatively, in the context of coordinated therapy among a cluster of several medical devices, all of the involved medical devices may be expected to deliver therapy so the HCP-accessible interface of the external computing device may be used to designate them all as medical devices that, subject to worst-case resets, may continue administering an output even if doing so was only feasible in a comparatively remedial manner (e.g., using a downgraded mode support condition or reduced feature support offerings).

Accordingly, the processor is further configured such that if it identifies that at least one pre-defined RAM application has become corrupted in an irreparable manner (e.g., critical signature checks monitoring for bit flips are invalid and self-healing proves impossible), the operation of the processor controlling determination of therapy output and transmission of therapy output to the therapy signal generator is based on the data stored in the ROM sub-unit and the present state of the state element. The state element determines the type of operation of the processor within this “option of last resort” (i.e., if the at least one pre-defined RAM application has become corrupted in an irreparable manner). The number of states that may be adopted by the state element determines the number of processing modi/branches that may be carried out by the processor in this situation, wherein the minimum number of processing modi/branches is two. In the latter case the first state of the state element may be “0” and the second state of the state element may be “1”. Additionally, the data stored in the ROM sub-unit, i.e., the preconfigured worst-case program stored in the ROM, are/is used by the processor to adapt the operation of the processor to the specific needs of the medical device and the patient to provide a therapy in one of the states. With other words: In the case where a particular medical device switches from a normal state of operation to a worst-case reset condition (i.e., typically one where the implant's RAM application cannot be started), the device may nominally land in a ROM-directed state of operation.

In one embodiment, the processor is configured such that if the first state is the present state of the state element the processor inhibits determination of therapy output and if one second state is the present state of the state element the processor determines therapy output based on the data stored in the ROM sub-unit and transmits the determined therapy output to the therapy signal generator. This behavior is also called ROM-directed behavior. In this embodiment, the ROM sub-unit needs to be configured such that it may realize at least two modi of operation at a worst-case reset condition, at least a first modus where determination of a therapy output is inhibited and a second modus where a minimum therapy support is provided to the patient based on the data stored in the ROM sub-unit.

In an alternative and likely preferred embodiment, the processor is configured such it determines therapy output based on the data stored in the ROM sub-unit, wherein the processor is further configured such that if the first state is the present state of the state element it inhibits transmission of the determined therapy output to the therapy signal generator and if one second state is the present state of the state element the processor transmits the determined therapy output to the therapy signal generator. With regard to this embodiment, the ROM sub-unit design may be structured in ways that require no assessment/referencing of content resident in outside-of-ROM memory locations such that the resultant behavior of the implant is ultimately arbitrated by wholly independent hardware-enabled switch conditions. In such conditions a “one-size-fits-all” ROM sub-unit configuration may be adopted that would, akin to legacy products assume that always administering therapy was the correct and safe behavior, but the hardware-enabled switch conditions would stand downstream of such settings to determine whether or not the ROM sub-unit data (settings) ultimately resulted in the delivery of therapy to the patient.

With regard to an ILP or a conventional pacemaker, the therapy signal provided by the pacing signal generator may realize a nominal (e.g., 60 ppm) pacing rate.

In one embodiment, the second memory sub-unit additionally comprises a pre-defined security memory sub-section and the processor is configured such that the determination of the therapy output is additionally based on at least one parameter derived from the security memory sub-section if one second state is the present state of the state element. At least one parameter for therapy which is stored in a pre-defined security memory section having a higher operational reliability and accuracy than other any other memory section, for example, one or more registers specifically designed to minimize the likelihood of single-event upsets (SEU) (e.g., bit flip transitions associated with cosmic ray bombardment) or a RAM location with affiliate redundancies. Such a security memory section may store at least one parameter that is used during normal operation of the medical device, i.e., not in a worst-case reset condition, and may therefore adapt to the specific needs of the specific patient between follow-up. In one embodiment related to a pacemaker or an ILP as medical device, at least one parameter derived from the pre-defined security memory sub-section is the pulse width and/or the pulse amplitude. This embodiment provides capacity for a medical device that is being directed to render therapy (that has furthermore been subjected to worst-case reset conditions) provisioning a means to configure the output amplitude and pulse width based on a recent history of conditions previously having been known to confer myocardial pacing capture. This may be adapted similarly with regard to other medical devices, too.

The detector of the implantable medical device may alternatively or additionally comprise an accelerometer, a vibration sensor, an acoustic sensor (including ultrasound) and/or any other mechanical, electric and/or magnetic sensor that is capable to detect the activity of the patient dependent on time (i.e., a motion sensor), e.g., whether the patient moves or moves not, for example, lies, sleeps, sits, moves fast or slowly, including exercising. Such motion sensor collects the activity signals of the patient and transforms them into electrical signals. Further, the detector may digitize analog signals, filter them and/or smooth them in order to reduce signal noise. Some pre-processing steps may be provided by the detector, as well. The determined signal produced by the detector may be transmitted to the processor directly.

The implantable medical device may comprise further modules such as a power supply (e.g., a battery). The components/units of the implantable medical device described above and below may be located within a hermetically sealed housing.

At least the above objective is further solved by a system comprising at least one implantable medical device of any of the previous claims and an external computing device, for example, a programmer, configured to transmit information to the at least one implantable medical device. As indicated above, the HCP may instruct any single medical device of a multitude of above-described medical devices resident within a patient regarding its “go forward” therapy (or no therapy) state using the external computing device-especially the “go forward” therapy (or lack thereof) subject to worst-case reset conditions.

In one embodiment of the system, the system comprises at least two medical devices and the external computing device is configured to transmit information to a specific one of the at least two medical devices based on a unique identifier which is assigned to each of the at least two medical devices prior implantation, for example, a serial number. In one embodiment one of the at least two medical devices is intended to replace another one of the at least two medical devices with regard to the provisioning of patient therapeutic/diagnostic support.

Due to limited data transmission capacity of deeply implanted medical devices (e.g., ILP), if two medical devices are within the range of the external computing device, instead of transmitting the (longer) serial number of each device in every communication as the unique identifier, the serial number may be shortened into a shortened unique identifier. Said shortened unique identifier may be as simple as designating the first leadless cardiac pacemaker as device 1 and the second leadless cardiac pacemaker as device 2 or may be any single-digit number, e.g., the last number, of the serial number, wherein the shortened unique identifier has to be defined such that there is a different number for each of the at least two medical devices.

At least the above object is further solved by an operation method of an implantable medical device, for example, an implantable leadless pacemaker, comprising a processor, a therapy signal generator, for example, a pacing signal generator, as well as a communication unit and a memory unit, wherein the memory unit is configured to exchange data with the processor, wherein the memory unit comprises a ROM sub-unit, a second memory sub-unit and a state element, wherein the state element is set to one state of a first state and at least one second state, wherein the second memory sub-unit comprises a RAM, wherein the communication unit receives information comprising a state input information from an external computing device and transmits at least one data corresponding to this information to the processor, wherein the processor sets the state of the state element based on the received data corresponding to state input information, wherein if the processor identified that at least one pre-defined RAM application has become corrupted in an irreparable manner, the processor is operated controlling determination of therapy output and transmission of therapy output to the therapy signal generator based on the data stored in the ROM sub-unit and the present state of the state element. The operation method may be regarded as computer implemented method as the operation method is executed by the processor of the implantable medical device.

In one embodiment of the method, if the first state is the present state of the state element the processor inhibits determination of therapy output and if one second state is the present state of the state element the processor determines therapy output based on the data stored in the ROM sub-unit and transmits the determined therapy output to the therapy signal generator.

In one alternative embodiment of the method, the processor determines therapy output based on the data stored in the ROM sub-unit, wherein if the first state is the present state of the state element the processor inhibits transmission of the determined therapy output to the therapy signal generator and if one second state is the present state of the state element the processor transmits the determined therapy output to the therapy signal generator.

In one embodiment of the method, the second memory sub-unit additionally comprises a pre-defined security memory sub-section and the processor determines the therapy output additionally based on at least one parameter derived from the security memory sub-section if one second state is the present state of the state element.

In one embodiment of the method, at least one parameter derived from the pre-defined security memory sub-section is the pulse width and/or the pulse amplitude, in particular, if the implantable medical device is an ILP.

The above embodiments of the operation method have the same advantages as the above medical device. Embodiments of the medical device indicated above may be realized in the operation method analogously. It is referred to the above explanation of the medical device in this regard.

The above method is, for example, realized as a computer program which comprises instructions which, when executed, cause the processing unit (processor) to perform the steps of the above method (to be executed by the cardiac pacemaker, in particular at its processor) which is a combination of above and below specified computer instructions and data definitions that enable computer hardware to perform computational or control functions or which is a syntactic unit that conforms to the rules of a particular programming language and that is composed of declarations and statements or instructions needed for the above and below specified function, task, or problem solution.

Furthermore, a computer program product is disclosed comprising instructions which, when executed by the processor, cause the processor to perform the steps of the above defined method. Accordingly, a computer readable data carrier storing such computer program product is disclosed.

The above-described medical device, operation method, system, computer program and computer program product maximize patient safety subject to worst-case reset behaviors in multi-device systems. Further, in specific change-out contexts where an “old” medical device is set to a disabled state and a “new” medical device is set to a therapy condition, the above solution offers a system and method for ensuring the “old” device avoids administering therapy output subject to worst-case resets. Were the “old” device to render such output, it could risk inhibiting the “new” device while simultaneously failing to capture the patient's condition. The above-described subject matter therefore assists in sidestepping the potential to leave patients without therapy support in worst-case reset configurations where explanting an expiring medical device is not clinically feasible. In the specific context of future envisionable systems where a group of medical devices coordinate their behaviors to render a resultant therapy, the above described subject matter provides a means to guarantee safe device/patient interactions (i.e., always rendering a safe output subject to worst-case resets—as notionally adopted in legacy products) without clobbering the essential behaviors and safety needs outlined above. In cases where therapy output is expected from a medical device and that same device witnesses a worst-case reset, the output offered by the device finds a balance between grotesque taxation of the limited power support available in a miniaturized medical device and also offering an expected capture response to facilitate viable therapy support till the next follow-up.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawing, wherein:

FIG. 1 shows a first embodiment of two ILPs within a cross section of a patient's heart,

FIG. 2 depicts a functional block diagram of one ILP shown in FIG. 1,

FIG. 3 shows an enlarged side view of one ILP depicted in FIG. 1,

FIG. 4 shows a flowchart of a first embodiment of an operation method of one ILP shown in FIG. 1, and

FIG. 5 shows a flowchart of a second embodiment of an operation method of one ILP depicted in FIG. 1.

### DETAILED DESCRIPTION

In the following the present invention is described with regard to a system comprising a programmer as an external computing device and two ILPs as implantable medical devices. It may analogously be realized for a system comprising other implantable medical devices or medical devices of different number or other external computing device, as well.

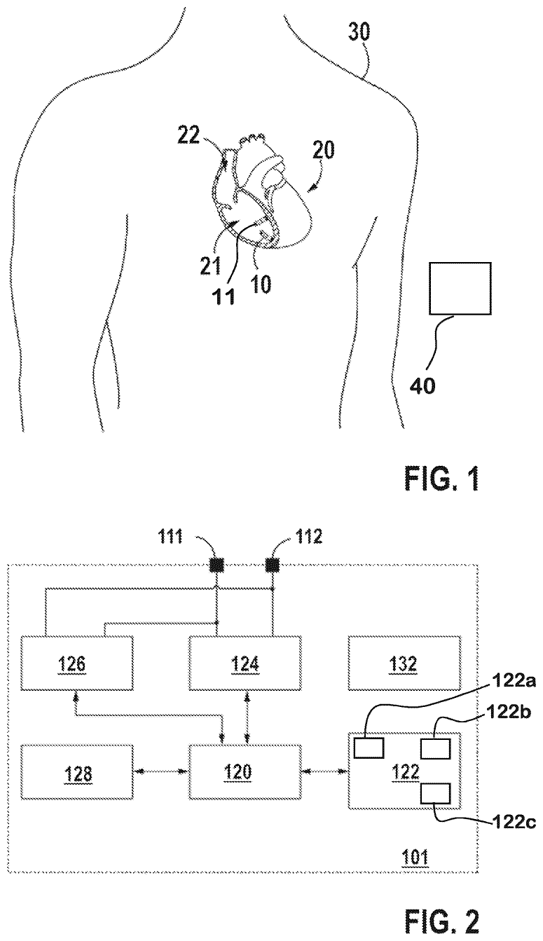

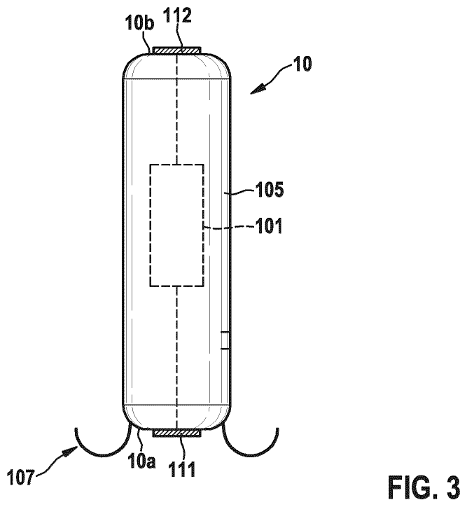

FIG. 1 shows an example first leadless ventricular pacemaker (ILP) 10 and an example second ILP 11 implanted within the heart 20 of a patient 30. The exemplary first ILP 10 is depicted in FIG. 3 in an enlarged view. The second ILP 11 has a similar construction and operates in the same way as the first ILP 10.

The first ILP 10 has a distal end 10a and a proximal end 10b and may be configured to be implanted within the right ventricle 21 of the heart 20 and pace this ventricle, sense intrinsic ventricular depolarizations and potentially also intrinsic atrial depolarizations (e.g., the right atrium 22), and inhibit ventricular pacing in response to detected intrinsic ventricular signal in VDD and VVI-based mode variants. A programmer 40 is used to program ILPs 10, 11 and retrieve data from ILPs 10, 11 using wireless communication such as WLAN.

FIG. 2 shows a functional block diagram of circuitry 101 of the ILP 10 configured for implantation within ventricle 21 (FIG. 1). The circuitry 101 of ILP 10 comprises a clocked processor 120, counters for the clock signals, a memory unit 122, a pacing signal generator 124, a detector unit 126, a communication unit 128 for communication with the programmer 40, and a power source 132. The power source 132 may be electrically connected to one or more of the other components 120, 122, 124, 126, 128 (not shown in FIG. 2) and may include a battery, e.g., a rechargeable or non-rechargeable battery. The power source provides electrical energy to all units and components of the ILP 10, in particular to all units mentioned above and is therefore electrically connected to these units and components. The mentioned units included in ILP 10 represent their respective functionality. Similar or identical units and functionality may also be included in the ILP 10. Units of the pacemaker of present disclosure may include any discrete and/or integrated electronic circuit components that implement analog and/or digital circuits capable of producing the functions attributed to the units herein. For example, the units may include analog circuits, e.g., amplification circuits, filtering circuits, and/or other signal conditioning circuits. The units may also include digital circuits, e.g., combinational or sequential logic circuits, memory devices, etc. The units may be further realized using integrated dedicated hardware logic circuits. The memory unit 122 may include any volatile, non-volatile, magnetic, or electrical media mentioned above. Furthermore, the processor 120 may include instructions that, when executed by one or more processing circuits, cause the units to perform various functions attributed to these units herein. The functions attributed to the units or component herein may be embodied as one or more processors, hardware, firmware, software, or any combination thereof. Depiction of different features as units or components is intended to highlight different functional aspects, and does not necessarily imply that such units must be realized by separate hardware or software components. Rather, functionality associated with one or more units or components may be performed by separate hardware or software components, or integrated within common or separate hardware or software components. Memory unit 122 may store computer-readable instructions that, when executed by processor 120, cause processor 120 to perform the various functions attributed to processor 120 herein. Further, memory unit 122 may store parameters for these functions, e.g., pacing signal parameters, conditions and thresholds described above and below. The pacing instructions and pacing signal parameters, conditions and thresholds may be updated by the programmer using the communication unit 128. The communication unit 128 may comprise an antenna or a transceiver.

The processor 120 may communicate with pacing signal generator 124 and detector 126 thereby transmitting signals. Pacing signal generator 124 and detector 126 are electrically coupled to electrodes 111, 112 of the ILP 10. Detector 126 is configured to monitor signals from electrodes 111, 112 in order to detect the electrical activity of heart 20. Further, the detector 126 may include a motion sensor, for example, an accelerometer or any other motion sensor described above. The motion sensor collects a time-dependent motion signal as described above and transmits this signal to the processor 120. Pacing signal generator 124 is configured to deliver electrical stimulation signals to ventricle 21 via electrodes 111, 112. Processor 120 may control pacing signal generator 124 to generate and deliver electrical stimulation to ventricle 21 via electrodes 111, 112. Electrical stimulation may include pacing pulses.

The electrode 112 is located where a mechanical hitch resides which means, in a preferred or practical built embodiment, the electrode 112 is not easily be located at the proximal most end (as shown in FIG. 3). The better approach would be to present a ring electrode near the proximal end, but not at the absolute extreme terminus of the device.

Processor 120 may control pacing signal generator 124 to deliver electrical stimulation therapy according to one or more therapy programs including pacing parameters, which may be stored in memory unit 122.

Detector 126 may further include circuits that acquire time-dependent electrical signals (e.g., electric depolarization and repolarization signals) from the heart including intrinsic cardiac electrical activity. Detector 126 may filter, amplify, and digitize or otherwise preprocess the acquired electrical signals of the heart chambers contractions. Processor 120 may receive the intrinsic electrical signals generated by detector 126 and perceive the intrinsic atrial signals and intrinsic ventricular signals of the patient's heart.

Processor 120 may assess the intrinsic atrial signal and the intrinsic ventricular signal received from the detector 126 and is configured to determine the intrinsic interval of two consecutive ventricular signals (at least one is intrinsic) or an intrinsic AV interval (interval between an intrinsic atrial signal and the subsequent ventricular signal).

ILP 10 may include a housing 105, fixation tines 107, and the electrodes 111, 112. The housing 105 may have a pill-shaped cylindrical form factor in some examples. Fixation tines 107 are configured to connect (e.g., anchor) ILP 10 to heart 20. Fixation tines 107 may be fabricated from a shape memory material, such as Nitinol. In some examples, fixation tines 107 may connect ILP 10 to heart 20 within one of the chambers of heart 20. For example, as illustrated and described herein with respect to FIG. 1, fixation tines may be configured to anchor ILP 10 to heart 20 within right ventricle 21. Although ILP 10 includes a plurality of fixation tines 107 that are configured to anchor ILP 10 to cardiac tissue in the right ventricle, it is contemplated that a pacemaker according to the present disclosure may be fixed to cardiac tissue in other chambers of a patient's heart 20 using other types of fixation mechanisms.

ILP 10 may include two electrodes 111, 112, although more than two electrodes may be included on a pacemaker in other examples. Electrodes 111, 112 may be spaced apart a sufficient distance to be able to detect various electrical signals generated by the heart 20, such as P-waves generated by atria and QRS complex generated by ventricles. For example, the first electrode 111 is located at the distal end 10a of the ILP 10 and the second electrode 112 is located at the proximal end 10b of the ILP 10. The housing 105 houses electronic components (circuitry 101) of ILP 10. Electronic components may include any discrete and/or integrated electronic circuit components that implement analog and/or digital circuits capable of producing the functions attributed to ILP 10 described above and below.

The communication unit 128 of circuitry 101 may enable ILP 10 to communicate with other electronic devices, such as the programmer 40 or other external patient monitor. In some examples, the housing 105 may house an antenna or transceiver for wireless communication. Housing may also include the power source 132.

The processor 120 may be adapted to control pacing of the right ventricle 21 in the VDD mode based on the intrinsic atrial signal containing atrial contractions and the intrinsic ventricular signal indicating ventricular contractions. Alternatively, a supplementary or alternative mode may be used by the processor 120 if the VDD mode turns out to be not appropriate for the individual patient in the present situation or is relevant to a specific device variant.

The memory unit 122 comprises a ROM sub-unit 122a, a second sub-unit 122b comprising a RAM and a state element 122c. The state element 122c may adopt two states, namely, “0” and “1”. Parameters for the determination and application of a suitable pacing signal by the processor 120 are stored in the ROM sub-unit 122a and the second sub-unit 122b, e.g., in the RAM. The second sub-unit 122b, in particular the RAM, comprises a pre-defined security memory sub-section, e.g., a register that is specifically designed to minimize the likelihood of bit flip transition (DICE register) or a RAM location with affiliate redundancies.

The processor 120 provides the synchronization and timing for ventricular pacing and inhibition based on clinical programming and the sensed intrinsic timing from the heart (e.g., sensed electrical signals from the heart) provided by detector 126. Additionally, the detector 126 may provide detected time-dependent motion signals and transmit them to the processor 120.

In order to control the ILPs 10, 11 the programmer 40 employs an interface within the programmer's environment through which the HCP may instruct specific, targeted ILPs 10, 11 to be designated as ones essential for administering therapy or as ones that should be disabled and henceforth deny stimulation output. Each ILP 10, 11 is addressed using a unique identifier that was assigned to the ILP 10, 11 prior to implantation. In the context of change-out conditions such instruction could simply amount to indicating which ILP was the “old” ILP (e.g., ILP 10) and which was the “new” replacement ILP (e.g., ILP 11). In a nominal case the “old” ILP 10 is transitioned toward a deactivated state where it would not be expected to render therapy at later points in time and the “new” ILP 11 is designated as one intended to provide pacing henceforth. Alternatively, in the context of coordinated therapy among a cluster of ILPs 10, 11, all of the involved ILPs would be expected to deliver therapy so the HCP-accessible interface would designate them all as ILP that, subject to worst-case resets would continue administering an output even if doing so was only feasible in a comparatively remedial manner (e.g., using a downgrade mode support condition or reduced feature support offerings). The interface of the programmer further allows the HCP to set/change the state of the state element 122c. The communication unit 128 receives this information and any other parameters provided by the programmer 40 and transmits these data to the processing unit 120. The processing unit 120 manages storing these data in the respective units/areas of the memory 122.

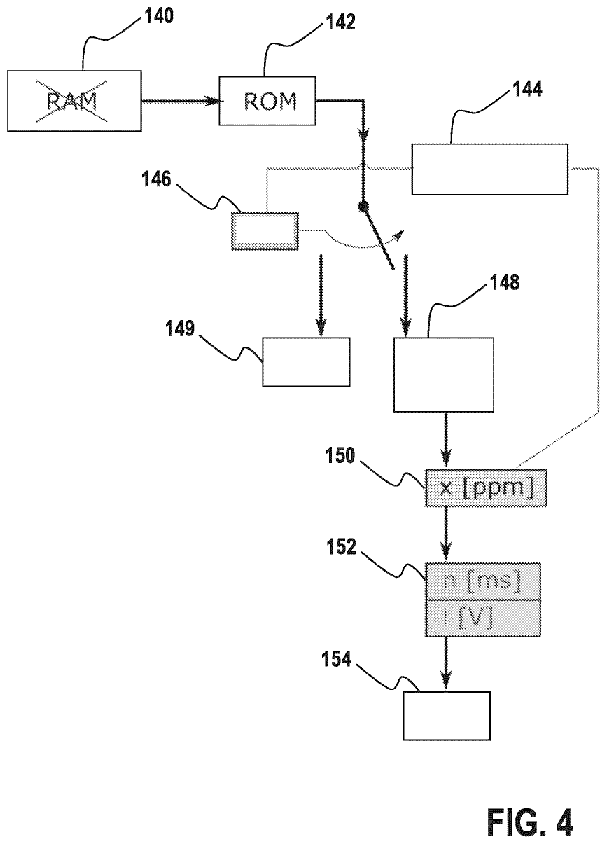

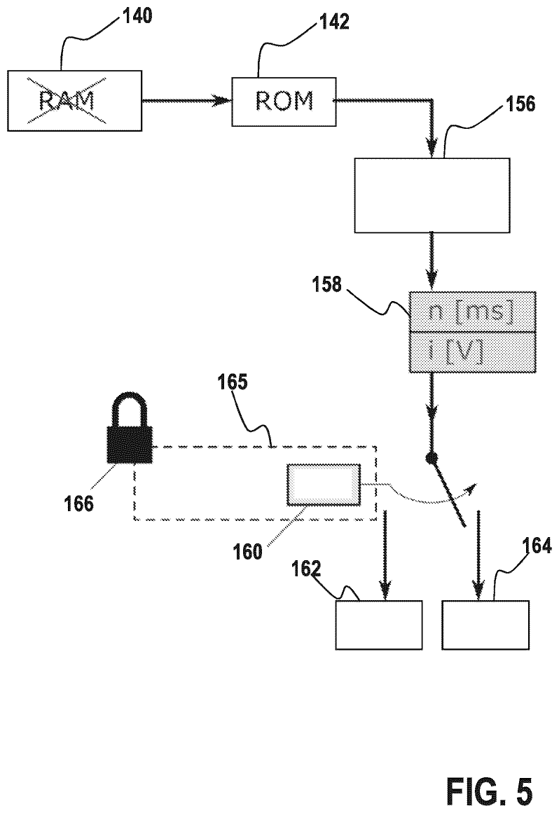

In the case where one of the ILPs 10, 11, for example, ILP 10, switches from a normal state of operation to a worst-case reset condition (i.e., typically one where the implant's RAM application cannot be started, symbolized by box 140 in FIG. 4 or 5), the ILP 10 nominally lands in a ROM-directed state of operation (box 142). Two embodiments of such operation is described with regard to the flowcharts shown in FIGS. 4 and 5 in the following.

The worst-case reset, ROM-directed behavior is initiated (step 142) in the first embodiment shown in FIG. 4 in such a manner that the present state is queried by the processor 120 from the state element 122c (step 144). Based on the received present state of the state element 122c the processor 120 executes (symbolized by box 146) either one of two ways of operation (boxes 148, 149). In the first way of operation (branch of FIG. 4 starting with box 148) the ROM is designed in a way where, as an element affiliated with its start-up, it assesses at least one parameter (e.g., pace rate) from the ROM 122a (see step 150) and at least one parameter stored in an outside-of-ROM memory location of the second sub-unit 122b which comprises a pre-defined security memory sub-section and uses the contents found in such security memory sub-section to prescribe aspects of the resultant ROM-directed behavior (e.g., pulse width and amplitude parameters, step 152). The respective therapy is determined by the processor 120 and transmitted to the pacing generator 124 to be administered (step 154). Therein settings for the amplitude and pulse width amount to values expected to support capture while also avoiding inclinations to overtax the limited on-board power resources. This approach demands that there be a degree of trust that the ROM can assign to the robustness and reliability of memory contents referenced in determining the behaviors of the device. This type (i.e., branch starting with box 148) of ROM-directed behavior is provided if the processor 120 identifies the state of the state element 122c as “1”.

In case the processor 120 derives from the state element 122c the present state value “0”, no therapy is delivered from the ILP 10 (see branch of FIG. 4 starting with box 149). The “old” ILP 10 is switched off because, for example, the “new” ILP 11 now provides pacing of the patient's heart similar to the ILP 10 as described above.

Alternatively, as shown in FIG. 5, a “one-size-fits-all” ROM configuration may be adopted after ROM directed behavior is initiated in step 142 as worst-case reset. This embodiment assumes that always determining and administering therapy by the processor 120 (see step 156) was the correct and safe behavior to a worst-case reset condition using pulse width and amplitude parameters again pulled from outside of ROM security-enhanced memory locations (see step 158). There is further a hardware-enabled switch condition downstream of such settings to determine whether or not the ROM settings and therapy determination ultimately resulted in the delivery of therapy to the patient. This is provided by a query to the state element 122c with regard to its present state (step 160), wherein the present state of the state element is either “0” or “1”. In case the present state of the state element 122c is value “1” the determined therapy signal is transmitted to the pacing generator 124 and applied to the patient (step 164). If, however, the processor 120 derives the value “0” from the state element 122c as the present state, the determined pacing signal is not transmitted to the pacing generator and therefore not applied thereby inhibiting pacing of the patient (step 162).

The boxes 146 and 160 of FIGS. 4 and 5 further symbolize that the state of the state element 122c may be set/changed prior to implantation or during a follow-up by the programmer 40. The programmer 40 may provide the HCP with a respective interface, wherein, as indicated above, the values to be stored in the state element 122c may be, for example, “0” and “1” thereby representing two different states. As shown in FIG. 5 and symbolized by box 165 and lock 166 a dedicated programmer 40 may be allowed to unlock the state element 122c for write access in order to increase operation safety of the ILP 10, 11.

With regard to the two embodiments shown in FIGS. 4 and 5, the ROM design may be structured in ways that require less or no assessment/referencing of content resident in outside-of-ROM memory locations such that the resultant behavior of the implant is ultimately arbitrated by largely (if not wholly) independent hardware-enabled switch conditions. Variants of this approach could embody options that either wholesale ignored any settings in outside-of-ROM memory locations as well as variants that simply accessed fewer such locations (for purposes of configuring the pulse duration and amplitude alone, for example).

The above embodiments ensure that a worst-case reset behavior that amounts to a condition where an active implantable medical device has lost its capacity to function as nominally intended for an implantable medical device such as an ILP 10, 11 in a bulk of active medical device resorts to having ROM govern the device's operation. Further, paths are provided to ensure that this “option of last resort” behavior (whether it is governed by ROM or, partially, other means) is aware of and directed by HCP intent as prescribed at the last follow-up to persist retaining any specific device in either a therapy or no therapy condition.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An implantable medical device, for example an implantable leadless pacemaker, comprising a processor, a therapy signal generator, for example a pacing signal generator, as well as a communication unit and a memory unit, wherein the memory unit is configured to exchange data with the processor, wherein the memory unit comprises a ROM sub-unit, a second memory sub-unit and a state element, wherein the state element is configured to be set to one state of a first state and at least one second state, wherein the second memory sub-unit comprises a RAM, wherein the communication unit is configured to receive information comprising a state input information from an external computing device and to transmit at least one data corresponding to this information to the processor, wherein the processor is configured to set the state of the state element based on the received data corresponding to state input information, wherein the processor is further configured such that if it identified that at least one pre-defined RAM application has become corrupted in an irreparable manner, the operation of the processor controlling determination of therapy output and transmission of therapy output to the therapy signal generator is based on the data stored in the ROM sub-unit and the present state of the state element.

2. The medical device of claim 1, wherein the processor is configured such that if the first state is the present state of the state element the processor inhibits determination of therapy output and if one second state is the present state of the state element the processor determines therapy output based on the data stored in the ROM sub-unit and transmits the determined therapy output to the therapy signal generator.

3. The medical device of claim 1, wherein the processor is configured such it determines therapy output based on the data stored in the ROM sub-unit, wherein the processor is further configured such that if the first state is the present state of the state element it inhibits transmission of the determined therapy output to the therapy signal generator and if one second state is the present state of the state element the processor transmits the determined therapy output to the therapy signal generator.

4. The medical device claim 1, wherein the second memory sub-unit additionally comprises a pre-defined security memory sub-section and the processor is configured such that the determination of the therapy output is additionally based on at least one parameter derived from the security memory sub-section if one second state is the present state of the state element.

5. The medical device of claim 4, wherein at least one parameter derived from the pre-defined security memory sub-section is the pulse width and/or the pulse amplitude.

6. A system comprising at least one implantable medical device of claim 1 and an external computing device, for example a programmer, configured to transmit information to the at least one medical device.

7. The system of claim 6, wherein the system comprises at least two medical devices and the external computing device is configured to transmit information to a specific one of the at least two medical devices based on a unique identifier which is assigned to each of the at least two medical devices prior implantation.

8. An operation method of an implantable medical device, for example an implantable leadless pacemaker, comprising a processor, a therapy signal generator, for example a pacing signal generator, as well as a communication unit and a memory unit, wherein the memory unit is configured to exchange data with the processor, wherein the memory unit comprises a ROM sub-unit, a second memory sub-unit and a state element, wherein the state element is set to one state of a first state and at least one second state, wherein the second memory sub-unit comprises a RAM, wherein the communication unit receives information comprising a state input information from an external computing device and transmits at least one data corresponding to this information to the processor, wherein the processor sets the state of the state element based on the received data corresponding to state input information, wherein if the processor identified that at least one pre-defined RAM application has become corrupted in an irreparable manner, the processor is operated controlling determination of therapy output and transmission of therapy output to the therapy signal generator based on the data stored in the ROM sub-unit and the present state of the state element.

9. The method of claim 8, wherein if the first state is the present state of the state element the processor inhibits determination of therapy output and if one second state is the present state of the state element the processor determines therapy output based on the data stored in the ROM sub-unit and transmits the determined therapy output to the therapy signal generator.

10. The method of claim 8, wherein the processor determines therapy output based on the data stored in the ROM sub-unit, wherein if the first state is the present state of the state element the processor inhibits transmission of the determined therapy output to the therapy signal generator and if one second state is the present state of the state element the processor transmits the determined therapy output to the therapy signal generator.

11. The method of claim 8, wherein the second memory sub-unit additionally comprises a pre-defined security memory sub-section and the processor determines the therapy output additionally based on at least one parameter derived from the security memory sub-section if one second state is the present state of the state element.

12. The method of claim 11, wherein at least one parameter derived from the pre-defined security memory sub-section is the pulse width and/or the pulse amplitude.

13. A computer program product comprising instructions which, when executed by a processor, cause the processor to perform the steps of the method according to claim 8.

14. Computer readable data carrier storing a computer program product according to claim 13.
